(19) United States
(12) Patent Application Publication
(10) Pub. No.: US 2011/0104154 A1
(43) Pub. Date: May 5, 2011

(54) SINGLE NUCLEOTIDE POLYMORPHISMS AND GENES ASSOCIATED WITH AGE-RELATED MACULAR DEGENERATION

(75) Inventors: Abbot F. Clark, Fort Worth, TX (US); Edwin M. Stone, Iowa City, IA (US); Val C. Sheffield, Iowa City, IA (US); John Fingert, Iowa City, IA (US); Thomas L. Casavant, Iowa City, IA (US); Todd Scheetz, Iowa City, IA (US); Terry Braun, Iowa City, IA (US); A. Jason Grundstad, Oak Lawn, IL (US)

(73) Assignees: ALCON RESEARCH, LTD., Fort Worth, TX (US); IOWA RESEARCH FOUNDATION, UNIVERSITY OF, Iowa City, IA (US)

(21) Appl. No.: 12/915,768

(22) Filed: Oct. 29, 2010

Related U.S. Application Data
(60) Provisional application No. 61/256,464, filed on Oct. 30, 2009.

(51) Int. Cl. A61K 39/395; G01N 33/50; C40B 40/06; A61P 27/02

(52) U.S. Cl. 424/133.1; 436/94; 506/16

(57) ABSTRACT

The invention provides genes and polymorphisms associated with AMD, and methods for diagnosing an increased risk of AMD in a patient who has at least one of the AMD-associated polymorphisms as provided.

### CROSS-REFERENCE TO RELATED APPLICATION

This application claims priority under 35 U.S.C. §119 to U.S. Provisional Patent Application No. 61/256,464, filed Oct. 30, 2009, the entire contents of which are incorporated herein by reference.

### FIELD OF THE INVENTION

The invention relates to genes and polymorphisms, including single nucleotide polymorphisms (SNPs) and clusters of SNPs, associated with AMD. In addition, the invention relates to methods for diagnosing an increased risk for AMD in a patient who has at least one of the AMD-associated polymorphisms as provided.

### BACKGROUND

Age-related macular degeneration (AMD) is a debilitating, blinding disease that affects the macular or central area of the retina responsible for high-acuity vision and is the leading cause of irreversible vision loss in the elderly. Both genetic and environmental factors are known to play a role in the development of AMD. For example, smoking, lipid intake and age are known risk factors for the development of AMD. The two forms of AMD, dry-AMD and wet-AMD, affect more than 11 million individuals in the US. Dry-AMD occurs in 80% of AMD patients and is characterized by the presence of cellular debris (drusen) in Bruch's membrane under the retinal pigment epithelium (RPE), irregularities in the RPE pigmentation, or geographic atrophy. Wet-AMD, occurring in the remaining 20% of AMD patients, is characterized by choroidal neovascularization and/or detachment of the RPE. Extracellular matrix abnormalities in the eyes of AMD patients have also been implicated.

The diagnosis of dry age-related macular degeneration is defined by the presence of drusen under the RPE and is seen in the early stages of disease. Drusen are small yellowish extracellular deposits composed of protein, lipid, and cellular debris. Drusen usually are confluent with significant pigment changes and accumulation of pigment in the posterior pole. RPE often appears atrophic with an easier visualization of the underlying choroidal plexus. In advanced stages of dry AMD, these focal islands of atrophy coalesce and form large zones of atrophy with severely affected vision. Wet AMD is defined by the presence of choroidal neovascularization and may include RPE elevation, exudate, or subretinal fluid.

A number of research groups have been intensively searching for genes associated with and responsible for the development of AMD. The Edwards study (Edwards et al., 2005, Science 308:421-424) involved scientists at UT Southwestern, Boston University and Sequenom. They performed SNP genotyping through the ARMD1 locus initially using 24 SNPs, then further refining the area with additional SNPs, in 2 case controlled populations (224 AMD patients and 134 controls in the first population; 176 cases and 68 controls in the second). They report that the individuals with one copy of the Y402H SNP in complement factor H had a 2.7× increased risk of developing AMD. This single SNP appears to account for 50% of AMD in their populations.

The Haines study (Haines et al., 2005, Science 308:419-421) was a collaborative study done at Vanderbilt University and Duke University. Similar to the Edwards study, Haines and colleagues SNP genotyped their 2 AMD populations across the ARMD1 locus. Their populations consisted of 182 AMD families and a case control population of 495 AMD patients and 185 controls. They initially used 44 SNPs to screen across the ARMD1 locus, then refined their search using additional SNPs. In their overall AMD population they found that patients heterozygous (bearing one copy) of the Y402H SNP in CFH had a 2.45 elevated risk for AMD, while homozygous individuals (having both copies of this SNP) had a 3.33 fold risk. The risk was even higher for those patients with neovascular (wet) AMD (3.45 in heterozygous and 5.57 in homozygous). They estimate that this SNP is responsible for 43% of AMD in their population.

The Klein study (Klein et al., 2005, Science 308:385-389) involved scientists at Rockefeller University, Yale University, The National Eye Institute (NEI), and EMMES Corporation. Unlike the previous 2 studies, the Klein group performed a genome-wide SNP genotype screen of 96 AMD patients and 50 controls using >116,000 SNPs. All the individuals in this study were clinically well-defined from the AREDS study population. The Klein group independently mapped the AMD susceptibility locus to chromosome 1q (the same regions as ARMD1) and identified the Y402H SNP in CFH as the risk allele. Individuals bearing one copy of this allele (heterozygous) had a 4.6× elevated risk, while individuals bearing this SNP on both chromosomes (homozygous) had a 7.4× elevated risk for AMD.

The Hageman study (Hageman et al., 2005, Proc Natl Acad Sci USA 102:7227-7232) included patients from the University of Iowa and Columbia University. They based their analysis of CFH on their previous studies that identified complement in the formation of Drusen and previous linkage analysis studies that identified the chromosomal locus 1q25-32. The Hageman group analyzed 900 AMD patients and 400 matched controls for SNPs within the CFH gene. In addition to the Y402H variant identified in the previous publications, Hageman et al. identified other AMD risk variants, such as 162V, intervening sequences 1, 2, 6, and 10, A307A, and A473A.

Confirmation of the Edwards, Haines, Klein, and Hageman findings may be found in at least three follow-up studies by Conley et al. (2005), Zareparsi et al. (2005) and Souied et al. (2005). Conley et al. (2005) identified a significant association of the Y402H variant with AMD patients in 796 familial and 196 sporadic AMD cases relative to 120 unaffected, unrelated controls. Zareparsi et al., (2005) found that the T>C substitution in exon 9 (Y402H) was associated with AMD in their single center study population. Souied et al. (2005) extended the original findings of the Y402H polymorphism association with AMD in the North American populations to the European (French) AMD population. Souied et al. examined 60 sporadic and 81 familial AMD cases and found a significant association of the Y402H polymorphism with AMD relative to 91 healthy controls. Thus, it appears that the Y402H polymorphism association with AMD is a reproducible and generalized finding.

In spite of the studies mentioned above, there remains a need for methods of diagnosing AMD, and for identifying patients as being at risk for developing AMD or for progressing from dry-AMD to wet-AMD. Early diagnosis and identification of such risks can enable health care providers to determine proper treatment regimens and manage progression of disease. In particular, genetic-based diagnostic assays will enable clinicians to treat patients sooner than the current standard testing allows, and will allow patients who have a high risk for AMD, or a risk of their dry-AMD progressing to wet-AMD, to take preventive measures to save or minimize the loss of their eyesight.

### SUMMARY

The invention provides a number of genes and polymorphisms that are associated with AMD. The polymorphisms include single nucleotide polymorphisms (SNPs) and clusters of SNPs as identified in Table 1, 6, 8, 10 or 11 as provided herein. In certain aspects, an individual who has any of the polymorphisms identified in Table 1, 6, 8, 10 or 11 is diagnosed as having an increased risk for developing AMD, or may be diagnosed as having AMD. In one aspect, the methods of the invention comprise identifying a SNP having a P-Value of less than E⁻⁴ as shown in Table 1, 6, 8, 10 or 11.

The invention also provides methods for diagnosing increased risk for AMD in a patient, the method comprising: (a) obtaining a biological sample containing nucleic acid from the patient; and (b) analyzing the nucleic acid to detect the presence or absence of any of the AMD-associated polymorphisms identified in Table 1, 6, 8, 10, or 11, wherein the presence of a AMD-associated polymorphism identified in Table 1, 6, 8, 10 or 11 indicates an increased risk for AMD. In one aspect, the AMD-associated polymorphism is a single nucleotide polymorphisms (SNPs) identified in Table 1, 6, 8, 10 or 11, a single nucleotide polymorphism cluster (SNP cluster) identified in Table 1, 6, 8, 10 or 11, or a plurality of SNPs and/or SNP clusters identified in Table 1, 6, 8, 10 or 11. In a particular aspect, the presence of at least two SNPs identified in Table 1 or at least two SNP clusters correlates with an increased risk for AMD.

In other aspects, allele-specific primers or allele-specific probes can be used to analyze the nucleic acid from a sample. In still other aspects, the analysis can comprise sequence analysis, denaturing gradient gel electrophoresis (DGGE), single-strand conformation polymorphism (SCCP), denaturing high performance liquid chromatography (DHPLC), microarrays, or restriction fragment length polymorphism (RFLP) analysis.

The invention also provides methods for treating a patient that has one or more of the AMD-associated polymorphisms described herein, the method comprising the step of administering to the patient an agent for treating AMD.

The invention further provides kits for indicating whether a patient has an increased risk for AMD, comprising: (a) at least one oligonucleotide that can identify an AMD-associated polymorphism identified in Table 1, 6, 8, 10 or 11; and (b) instructions for use. In certain aspects, a kit comprises a set of oligonucleotides, wherein the set comprises at least one pair of primers that can detect at least one of the polymorphisms identified in Table 1, 6, 8, 10 or 11. In other aspects, a set of oligonucleotides in a kit comprises a plurality of primer pairs, each of which can detect at least one single nucleotide polymorphism identified in Table 1, 6, 8, 10 or 11. In a further aspect, a kit comprises a plurality of primer pairs, each of which can detect at least one SNP cluster identified in Table 1, 6, 8, 10 or 11. In another aspect, a kit of the invention comprises a set of oligonucleotide probes, each of which can hybridize to a polymorphism identified in Table 1, 6, 8, 10 or 11. A kit of the invention can further comprise a microarray.

Specific preferred embodiments of the invention will become evident from the following more detailed description of certain preferred embodiments and the claims.

### DETAILED DESCRIPTION OF THE INVENTION

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of skill in the art, such as the Oxford Dictionary of Biochemistry and Molecular Biology (Ed. Anthony Smith, Oxford University Press, Oxford, 2004).

As used herein and unless otherwise indicated, the terms “a” and “an” are taken to mean “one”, “at least one” or “one or more”. Unless otherwise required by context, singular terms used herein shall include pluralities and plural terms shall include the singular.

In certain embodiments, the invention provides genes and polymorphisms that are associated with AMD.

As used herein, the term “polymorphism” refers to the occurrence of genetic variations that account for alternative DNA sequences and/or alleles among individuals in a population. The term “polymorphic site” refers to a genetic locus wherein one or more particular sequence variations occur. A polymorphic site can be one or more base pairs. For example, a “single nucleotide polymorphism (SNP)” is a polymorphism that occurs at a single nucleotide. As used herein, a “cluster” of SNPs refers to three or more SNPs that occur within 100 kilobases of each other in a particular polymorphic site, wherein all of the SNPs have a P-Value of less than E⁻⁴ (i.e. <1×10⁻⁴).

As used herein, a “AMD-associated polymorphism” refers to a SNP or SNP cluster that correlates with AMD, and the presence of which in an individual indicates an increased risk of developing AMD. AMD-associated polymorphisms include the SNPs and the SNP clusters identified in Table 1, 6 and 8.

Tables 1, 6, 8, 10 and 11 identify a number of SNPs and SNP clusters that are associated with AMD. Each cluster comprises at least three SNPs. The SNPs in each cluster are localized to a common genomic locus on a particular chromosome as indicated in the Table. Each cluster's genomic location is also identified by genomic starting and ending position. Each SNP is identified by SNP Accession Number as identified in the National Center for Biotechnology Information (NCBI) dbSNP database. Those of skill in the art can readily identify the reference sequences and the particular positions of the SNP within the reference sequences using the dbSNP database Accession Number. The nucleotide change associated with each SNP is shown in brackets in Table 2. The P-Value was determined as described in the Examples below. P-Values shown in columns 3, 4, and 5 in Table 1 represent the results of the confirmatory phase (Phase 2) described in the Examples below. P-Values of less than or equal to 0.05 in columns 3 and 4 were considered to have especially strong association with AMD.

TABLE 1

| | | | | |
|---|---|---|---|---|
| | | data.AMD- | data.AMD- | data.GLC- |
| SNP | P-Value | GLC | NL | NL |

Cluster 1
Chromosome: chr1, Genomic Start: 194664398, GenomicStop: 195393272

| | | | | |
|---|---|---|---|---|
| SNP_A-2171106 | 1.00E−99 | 5.56E−17 | 2.23E−15 | 0.17203321 |
| SNP_A-4206823 | 1.00E−99 | 4.40E−10 | 5.63E−16 | 0.47875855 |
| SNP_A-2198520 | 1.00E−99 | 2.74E−11 | 9.19E−18 | 0.44341399 |
| SNP_A-1960732 | 1.11E−16 | 2.63E−09 | 1.03E−11 | 0.97809667 |
| SNP_A-4290423 | 2.22E−16 | 7.46E−08 | 1.70E−09 | 0.91238736 |
| SNP_A-2197019 | 5.55E−16 | 8.12E−09 | 1.93E−12 | 0.79352025 |
| SNP_A-1784833 | 1.15E−12 | 3.48E−11 | 1.17E−11 | 0.51681387 |
| SNP_A-2120752 | 1.82E−11 | 3.09E−10 | 2.15E−07 | 0.0949576 |
| SNP_A-1951468 | 3.74E−11 | 1.31E−05 | 1.40E−06 | 0.87238086 |
| SNP_A-4210538 | 2.59E−08 | 1.12E−05 | 0.01093622 | 0.03942654 |
| SNP_A-1841011 | 2.67E−08 | 0.10642471 | 0.01768046 | 0.71064677 |
| SNP_A-2047711 | 1.88E−07 | 9.93E−05 | 7.34E−06 | 0.97000736 |
| SNP_A-1841540 | 3.04E−07 | 3.68E−06 | 5.07E−11 | 0.32178607 |
| SNP_A-2287102 | 3.98E−07 | 0.00403262 | 1.11E−09 | 0.02392035 |
| SNP_A-2257497 | 6.51E−07 | 8.91E−05 | 0.0029787 | 0.21479681 |
| SNP_A-1955282 | 6.91E−06 | 0.00119588 | 8.61E−05 | 0.90958025 |
| SNP_A-1781581 | 2.03E−05 | 1.79E−05 | 0.00084951 | 0.19893448 |
| SNP_A-1802047 | 3.53E−05 | 2.48E−05 | 0.00314428 | 0.12422995 |
| SNP_A-2038830 | 4.88E−05 | 0.19820703 | 0.11296869 | 0.9757567 |
| SNP_A-2249917 | 5.03E−05 | 0.02773062 | 0.14075455 | 0.40046958 |
| SNP_A-1786975 | 5.26E−05 | 5.09E−05 | 0.00039801 | 0.39270702 |
| SNP_A-1952394 | 8.49E−05 | 0.00011248 | 0.05191733 | 0.04311259 |
| SNP_A-2211821 | 9.85E−05 | 0.32552631 | 0.08883968 | 0.01644031 |
| SNP_A-2265908 | 1.48E−04 | 0.01459117 | 0.7919689 | 0.03734277 |
| SNP_A-4242244 | 2.04E−04 | 0.07374127 | 0.47198707 | 0.28513318 |
| SNP_A-1901438 | 2.27E−04 | 0.00757898 | 0.22635581 | 0.13364456 |
| SNP_A-2046262 | 3.48E−04 | 0.00803068 | 0.03854226 | 0.43089699 |
| SNP_A-4245247 | 4.12E−04 | 0.12344706 | 0.31526159 | 0.01904617 |
| SNP_A-1919558 | 4.33E−04 | 0.00999446 | 0.45620018 | 0.00178264 |
| SNP_A-2272808 | 4.87E−04 | 0.01147223 | 0.60708556 | 0.00394346 |
| SNP_A-1960915 | 7.90E−04 | 0.0227205 | 0.53486281 | 0.10397591 |

Cluster 2
Chromosome: chr10, Genomic Start: 124139342,
GenomicStop: 124225345

| | | | | |
|---|---|---|---|---|
| SNP_A-1841655 | 1.00E−99 | 2.40E−13 | 5.40E−16 | 0.71685648 |
| SNP_A-2006209 | 1.11E−16 | 3.16E−13 | 4.51E−17 | 0.96790364 |
| SNP_A-2183447 | 1.12E−08 | 8.29E−05 | 1.96E−09 | 0.24695688 |
| SNP_A-2006207 | 1.38E−07 | 0.13436387 | 0.00035202 | 0.13916736 |
| SNP_A-2006206 | 2.19E−06 | 0.01796534 | 9.28E−06 | 0.17170543 |
| SNP_A-4240404 | 2.47E−04 | 0.66787209 | 0.01008365 | 0.09580169 |
| SNP_A-4230717 | 3.35E−04 | 0.34352577 | 0.59113505 | 0.16681682 |

The allele frequency for each SNP in Table 1 is shown in Table 2, where the “A” represents the first nucleotide shown in the brackets in the sequences from Table 1, and the “B” represents the second nucleotide shown in the brackets. For example, for SNP_A-2171106, “A” is G, “B” is T, and AA represents a patient having a GG haplotype, AB represents a patient having a GT haplotype, and BB represents a patient having a TT haplotype. The haplotypes from the AMD patients were compared with haplotypes from the AMD patients as described in the Examples below. As used herein, a “AMD-associated haplotype” is three or more high-risk SNPs in a cluster. A “high-risk” SNP has a P-Value of less than 1×10⁻⁴.

TABLE 2

| | | Allele Frequency | | | | | | |
|---|---|---|---|---|---|---|---|---|
| | | AMD | | | GLC | | | |
| SNP Name | Allele | AA | AB | BB | AA | AB | BB | P-Value |
| Cluster 1 | | | | | | | | |
| SNP_A-2171106 | [G/T] | 10 | 109 | 280 | 73 | 187 | 132 | 1.00E−99 |
| SNP_A-4206823 | [A/G] | 170 | 180 | 46 | 63 | 189 | 146 | 1.00E−99 |
| SNP_A-2198520 | [A/C] | 171 | 182 | 45 | 66 | 184 | 142 | 1.00E−99 |
| SNP_A-1960732 | [C/T] | 45 | 184 | 171 | 142 | 189 | 68 | 1.11E−16 |
| SNP_A-4290423 | [A/T] | 169 | 182 | 45 | 65 | 185 | 138 | 2.22E−16 |
| SNP_A-2197019 | [A/G] | 149 | 196 | 55 | 58 | 181 | 158 | 5.55E−16 |
| SNP_A-1784833 | [C/G] | 174 | 181 | 42 | 88 | 180 | 127 | 1.15E−12 |
| SNP_A-2120752 | [C/T] | 192 | 164 | 30 | 108 | 175 | 105 | 1.82E−11 |
| SNP_A-1951468 | [A/G] | 50 | 209 | 79 | 149 | 154 | 29 | 3.74E−11 |
| SNP_A-4210538 | [C/T] | 4 | 71 | 309 | 35 | 115 | 211 | 2.59E−08 |
| SNP_A-1841011 | [G/T] | 327 | 67 | 3 | 237 | 126 | 27 | 2.67E−08 |
| SNP_A-2047711 | [A/G] | 15 | 113 | 258 | 51 | 154 | 177 | 1.88E−07 |
| SNP_A-1841540 | [C/T] | 16 | 121 | 263 | 52 | 160 | 184 | 3.04E−07 |
| SNP_A-2287102 | [C/T] | 276 | 112 | 12 | 198 | 149 | 47 | 3.98E−07 |
| SNP_A-2257497 | [C/T] | 1 | 57 | 342 | 11 | 126 | 259 | 6.51E−07 |
| SNP_A-1955282 | [A/G] | 212 | 160 | 27 | 142 | 192 | 60 | 6.91E−06 |
| SNP_A-1781581 | [C/T] | 0 | 39 | 352 | 11 | 89 | 280 | 2.03E−05 |
| SNP_A-1802047 | [C/T] | 355 | 44 | 0 | 293 | 97 | 9 | 3.53E−05 |
| SNP_A-2038830 | [A/T] | 218 | 154 | 23 | 156 | 182 | 53 | 4.88E−05 |
| SNP_A-2249917 | [A/G] | 0 | 44 | 354 | 10 | 94 | 295 | 5.03E−05 |
| SNP_A-1786975 | [A/G] | 353 | 42 | 0 | 286 | 92 | 9 | 5.26E−05 |
| SNP_A-1952394 | [C/T] | 1 | 41 | 349 | 14 | 83 | 287 | 8.49E−05 |
| SNP_A-2211821 | [C/T] | 357 | 35 | 0 | 296 | 82 | 9 | 9.85E−05 |
| SNP_A-2265908 | [C/T] | 364 | 34 | 0 | 307 | 75 | 11 | 1.48E−04 |
| SNP_A-4242244 | [G/T] | 354 | 44 | 0 | 290 | 97 | 4 | 2.04E−04 |
| SNP_A-1901438 | [C/T] | 365 | 34 | 0 | 310 | 73 | 11 | 2.27E−04 |
| SNP_A-2046262 | [C/T] | 1 | 41 | 348 | 10 | 82 | 281 | 3.48E−04 |
| SNP_A-4245247 | [C/T] | 289 | 56 | 0 | 217 | 104 | 7 | 4.12E−04 |
| SNP_A-1919558 | [C/T] | 1 | 31 | 362 | 13 | 65 | 304 | 4.33E−04 |
| SNP_A-2272808 | [A/G] | 5 | 60 | 309 | 10 | 115 | 248 | 4.87E−04 |
| SNP_A-1960915 | [A/G] | 1 | 32 | 362 | 10 | 69 | 308 | 7.90E−04 |
| Cluster 2 | | | | | | | | |
| SNP_A-1841655 | [A/C] | 128 | 177 | 90 | 244 | 138 | 11 | 1.00E−99 |
| SNP_A-2006209 | [A/C] | 87 | 179 | 130 | 11 | 140 | 245 | 1.11E−16 |
| SNP_A-2183447 | [C/G] | 82 | 178 | 134 | 21 | 160 | 199 | 1.12E−08 |
| SNP_A-2006207 | [A/C] | 45 | 188 | 133 | 97 | 212 | 68 | 1.38E−07 |
| SNP_A-2006206 | [A/G] | 54 | 190 | 152 | 106 | 194 | 94 | 2.19E−06 |
| SNP_A-4240404 | [A/G] | 30 | 160 | 208 | 55 | 190 | 152 | 2.47E−04 |
| SNP_A-4230717 | [C/T] | 205 | 161 | 28 | 154 | 187 | 55 | 3.35E−04 |

In addition to the SNPs associated with AMD shown above, the following cluster was identified as having SNPs associated with glaucoma.

| Glaucoma Cluster | | | | | | | | |
|---|---|---|---|---|---|---|---|---|
| (Chromosome: chr6, | | | | | | | | |
| Genomic Start: 30911233, | | | | | | | | |
| Genomic Stop: 31030549) | | AMD | | | GLC | | | |
| SNP Name | Allele | AA | AB | BB | AA | AB | BB | P-Value |
| SNP_A-2196694 | [A/G] | 47 | 171 | 175 | 86 | 176 | 125 | 2.17E−04 |
| SNP_A-2056546 | [C/T] | 72 | 191 | 125 | 41 | 162 | 175 | 2.48E−04 |
| SNP_A-2256672 | [A/T] | 164 | 182 | 48 | 120 | 185 | 89 | 3.13E−04 |
| SNP_A-2182258 | [C/G] | 126 | 199 | 74 | 174 | 175 | 46 | 7.31E−04 |
| SNP_A-4256255 | [A/G] | 125 | 184 | 71 | 173 | 171 | 42 | 8.89E−04 |

The AMD-associated polymorphisms identified in Table 1, 6, 8, 10 or 11 correlates with AMD, as determined using the criteria discussed in the Examples herein, and are useful for diagnosing AMD and risk for developing AMD. According to the invention, the presence of one or more of the AMD-associated polymorphisms identified in Table 1, 6, 8, 10 or 11 indicates a high risk for developing AMD. The methods of the invention can be combined with ophthalmological examination, assessment of AMD risk factors (such as family history), and analysis of other polymorphisms associated with AMD that are known in the art.

In certain embodiments, the invention provides methods for determining the risk of a patient developing AMD. In certain embodiments, the methods of the invention involve screening a patient for the presence of certain allele specific polymorphisms associated with AMD. The methods of the invention are useful for routine screening of patients to determine their AMD risk, as well as for screening patients who may be suspected of having a high risk for developing AMD, such as a patient who has a family history of AMD.

As used herein, the term “patient” includes human subjects.

In certain embodiments, the methods for determining a patient's risk with respect to developing AMD involve analyzing nucleic acid from a biological sample of a patient for the presence of one or more allele specific polymorphisms associated with AMD. The presence of one or more of the AMD-associated polymorphisms indicates that a patient has an increased risk of developing AMD relative to a patient who does not have the AMD-associated polymorphism(s). In one embodiment, the presence of any of the polymorphisms identified in Table 1, 6, 8, 10 or 11 is indicative of an increased risk for developing AMD. A patient diagnosed as having an increased risk for AMD based on the presence of one or more of the polymorphisms identified in Table 1, 6, 8, 10 or 11 can take steps to reduce the risk of developing AMD, for example, by engaging in frequent ophthalmological examinations and/or increasing anti-oxidant intake and/or beginning one or more AMD treatments with an agent suitable for treating AMD.

Suitable AMD treatments include, but are not limited to, treatment with an agent that is an anti-VEGF molecule, such as LUCENTIS™ or MACUGEN®, a complement factor inhibitor, or Visudyne used with Photodynamic Therapy. Additional anti-VEGF molecules are known in the art, including molecules described in International Patent Application WO 03/012105, U.S. Pat. No. 7,148,342, U.S. Patent Application No. 2005/0233998, U.S. Patent Application No. 2005/0054596, U.S. Patent Application No. 2005/0222066, U.S. Pat. No. 7,517,864, U.S. Patent Application No. 2006/0094032, International Patent Application WO 2008/109377, U.S. Patent Application No. 2005/0255487, U.S. Patent Application No. 2007/0031844, U.S. Pat. No. 6,361,771, U.S. Pat. No. 7,115,257, U.S. Pat. No. 6,303,136 and U.S. Pat. No. 6,627,422, the disclosure of each of which is hereby incorporated by reference in its entirety. Suitable examples of complement inhibitors include compstatin and compstatin analogs as described, for example, in U.S. Pat. No. 6,319,897, International Patent Application WO 2004/026328, International Patent Application WO 2007/062249, the disclosure of each of which is hereby incorporated by reference in its entirety. Additional complement inhibitors are known in the art, as described, for example, in U.S. Patent Application No. 2002/0015957, the disclosure of which is hereby incorporated by reference in its entirety.

The phrase “increased risk” as used herein refers to an increased likelihood that a patient will develop AMD relative to individuals in the population without a polymorphism associated with AMD.

The term “biological sample” as used herein includes, but is not limited to, blood, saliva, cells from buccal swabbing, biopsies of organs (such as retina, kidney, liver, and skin), amniotic fluid, various other tissues and the like. Methods for purifying or partially purifying nucleic acids from a biological sample for use in diagnostic assays are well known in the art. The nucleic acid can be, for example, genomic DNA, RNA, or cDNA. Genomic DNA can be isolated, for example, from peripheral blood leukocytes using QIAamp DNA Blood Maxi Kits (Qiagen, Valencia, Calif.).

Numerous methods for analyzing a sample for polymorphisms are known in the art. For example, the methods of the invention can comprise allele specific primers, allele specific probes, sequence analysis, denaturing gradient gel electrophoresis (DGGE), single-strand conformation polymorphism (SCCP), denaturing high performance liquid chromatography (DHPLC), microarrays, and restriction fragment length polymorphism (RFLP) analysis. In some of these methods, and others, oligonucleotides are designed and employed to carry out the analysis.

The term “oligonucleotide” as used herein refers to a polymer of two or more nucleotides. An oligonucleotide may be DNA, RNA, or a combination of DNA and RNA, and may be single-stranded or double-stranded. Oligonucleotides can be chemically synthesized using methods well known to those of skill in the art. In certain embodiments, an oligonucleotide comprises one or more of the polymorphisms set forth in Table 1, 6, 8, or 11.

In one embodiment, the invention provides a set of allele specific oligonucleotides for diagnosing AMD or an increased risk for developing AMD. As used herein, an “allele specific oligonucleotide” can hybridize to one or more AMD-associated polymorphisms. In certain embodiments, the set comprises oligonucleotides for detecting at least two of the SNPs shown in Table 1, 6, 8, 10 or 11. In certain embodiments, the set comprises oligonucleotides for detecting all of the SNPs shown in Table 1, 6, 8, 10 or 11. In certain embodiments, the set comprises oligonucleotides for detecting at least one or more of the SNP clusters shown in Table 1, 6, 8, 10 or 11. In certain embodiments, the set comprises oligonucleotides for detecting at least two of the SNP clusters shown in Table 1, 6, 8, 10 or 11. In certain embodiments, the set comprises oligonucleotides for detecting all of the SNP clusters shown in Table 1, 6, 8, 10 or 11.

In certain embodiments, oligonucleotides of the invention are primers that can be used to detect the presence or absence of an allele specific polymorphism associated with AMD. In particular, the primers can be used to identify the presence or absence of a single nucleotide polymorphism (SNP) as set forth in Table 1, 6, 8, 10 or 11, or a SNP cluster as set forth in Table 1, 6, 8, 10 or 11.

The primers of the invention can be designed using techniques well known to those of skill in the art. For example, International Application WO 93/22456 describes methods for designing and using allele specific primers to detect polymorphisms.

Primer pairs can be designed to hybridize to regions adjacent to or including a particular polymorphic allele. A primer pair can be used to amplify nucleic acid from a biological sample. The amplified nucleic acid can be used in assays described herein to determine if the allele specific polymorphism is present in a patient's sample.

Amplification of DNA or RNA from the biological samples can be accomplished using standard polymerase chain reaction (PCR) and reverse transcription polymerase chain reaction (RT-PCR), for example. The amplified product can be sequenced to determine if a polymorphic site is present using various methods known to those skilled in the art. Other assays can also be used to analyze the amplified product, such as SSCP analysis, SNP-plex assay, and DHPLC analysis. SSCP analysis can be performed, for example, using Applied Biosystems SNP Assays-On-Demand quantitative PCR (Applied Biosystems, Foster City, Calif.).

In certain embodiments, the invention provides a set of primers that can detect one or more of the polymorphisms identified in Table 1, 6, 8, 10 or 11. For example, the set of primers can include a plurality of primer pairs, each of which can be used to amplify a nucleic acid that comprises a SNP identified in Table 1, 6, 8, 10 or 11 or a SNP cluster identified in Table 1, 6, 8, 10 or 11. Primers can be designed using methods well known to those of skill in the art based on the sequences surrounding a SNP or SNP cluster identified herein.

In other embodiments, the oligonucleotides of the invention are allele specific probes that can hybridize to a AMD-associated polymorphism. Methods for designing and generating probes are known in the art. See, for example, WO 89/11548 and EP 235726. Generally, probes are designed to distinguish between an allele that contains a polymorphism and an allele that does not. Hybridization conditions can be chosen to ensure specific hybridization of the probe to the polymorphic allele and not to the normal allele.

In certain embodiments, the invention provides a set of probes that can determine at least one polymorphism from Table 1, 6, 8, 10 or 11. For example, the set of probes can include a plurality of probes, each of which can hybridize to a SNP identified in Table 1, 6, 8, 10 or 11 or a SNP cluster identified in Table 1, 6, 8, 10 or 11.

In certain embodiments, a microarray for detecting AMD can be used in methods of the invention. The microarray can comprise one or more oligonucleotides of the invention or a complementary oligonucleotide thereof bound to a substrate. The biological sample or isolated nucleic acid molecules from the sample can be contacted with the microarray under suitable conditions for the oligonucleotides to hybridize to polymorphic regions in the nucleic acid from the sample. Unbound nucleic acid is washed away and bound nucleic acid is detected.

Analysis of nucleic acid from a sample can also be accomplished by using a SNP chip microarray, such as a SNP chip available from Affymetrix (Santa Clara, Calif.). SNP chips can be designed to contain a certain number of SNPs, such as any number (including all) of the SNPs identified in Table 1, 6, 8, 10 or 11 or any number (including all) of the SNP clusters identified in Table 1, 6, 8, 10 or 11.

A multi-plex PCR assay can also be used to analyze nucleic acid for polymorphisms. For example, a SNP-plex assay can be designed as described to detect one or more of the polymorphisms identified in Table 1, 6, 8, 10 or 11. SNP-plex assays are described, for example, in Sanchez et al., 2006, Electrophoresis 27:1713-24.

In one embodiment, the invention provides kits for AMD risk diagnosis. In certain embodiments, a kit of the invention comprises a set of allele specific oligonucleotides as provided herein to identify the presence or absence of one or more AMD-associated polymorphisms identified in Table 1, 6, 8, 10 or 11. For example, a kit comprises: a set of primers for amplifying polymorphic sites associated with AMD as described herein; a set of probes that can hybridize to polymorphic sites associated with AMD as described herein; and/or a microarray, such as a SNP chip, as described herein. Primers and probes can be readily and easily designed by those skilled in the art by reference to a sequence associated with the SNP accession numbers in Table 1, 6, 8, 10 or 11. Microarrays can also be easily and readily designed with oligonucleotides of the invention that correspond to sequences associated with the SNP accession numbers in Table 1, 6, 8, 10 or 11.

The references cited herein, to the extent that they provide exemplary procedural or other details supplementary to those set forth herein, are specifically incorporated by reference.

Those of skill in the art, in light of the present disclosure, will appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

### EXAMPLES

The following examples, including the experiments conducted and results achieved are provided for illustrative purposes only and are not to be construed as limiting the invention.

### Example 1

### Selection of SNPs Associated with AMD

A two-phase screening experiment was conducted to identify polymorphisms associated with age-related macular degeneration (AMD). Patients were evaluated and diagnosed with AMD by board certified and fellowship trained ophthalmologist using standard diagnostic criteria as follows.

Candidates for this project were selected from a pool of patients diagnosed with age-related macular degeneration (AMD) by a faculty ophthalmologist at the University of Iowa. Candidates' charts and photofiles were reviewed by a retinal expert with extensive experience in AMD and AMD trials. For inclusion in this study, a patient had to have either Category 3 or 4 AMD as defined by the Age-Related Treatment Trial in both eyes (Anand et al., 2000, Ophthalmology 2224-32; Age Related Eye Disease Study Research Group., 2001, AREDS Report No. 8. Arch. Ophthalmol., 1417-1436). For an eye to be classified as Category 3, it must have had at least one large (≧125μ) druse, or enough intermediate size (63-125μ) drusen that when the area occupied would be at least 0.5 disc area. A Category 4 eye was characterized as having advanced AMD defined as geographic atrophy of the retinal pigment epithelium (RPE) in the center of the fovea or choroidal neovascularization. Geographic atrophy of the RPE is defined in the AREDS as the presence of at least two of the three following characteristics: a circular area, sharply defined margins, and visible choroidal vessels. Signs of choroidal neovascularization include elevation of the retinal pigment epithelium, subretinal hemorrhage or fibrosis, serous retinal detachment, hard exudation and leakage of new vessels on fluorescein angiography. If a patient had Category 4 AMD in both eyes, at least one eye had to have at least one large drusen or a 0.5 disc area of intermediate drusen when added together.

Patients with evidence of myopic degeneration, chorioretinal scars in the macula, angioid streaks, or diabetic retinopathy consisting of more than five microaneursyms or hemorrhages were excluded from the study. The few patients who had equivocal findings, no photos, or poor quality photos that could not be evaluated were excluded from the study.

Patients were subdivided into groups based on past or present evidence of CNV up to and including their last follow-up examination. Patients with CNV in both eyes were placed in group one. Patients with CNV in only one eye were placed into group two. Patients with no CNV and were age 70 or older were placed into group three.

The AMD patients were rigorously evaluated for glaucoma to ensure that they could serve as a glaucoma-depleted control group. AMD patients were excluded from this study if they ever took medication to lower their intraocular pressure, had a history of any type of glaucoma or were a glaucoma suspect. They were also excluded if they had a cup to disc ratio in either eye of 0.5 or greater unless they had been evaluated by the glaucoma service at UIHC and deemed not to have glaucoma. Patients were also excluded if they ever had an intraocular pressure of 25 mm Hg or higher. Patients with an intraocular pressure of 22-24 mm Hg in either eye were included only if there were at least three other measurements that were below this mark.

A set of patients who were diagnosed as having primary open angle glaucoma served as controls. The AMD patients had no signs of POAG. The POAG patients did not have AMD and were determined not to be AMD suspects based on lack of suspicious drusen in their macula.

DNA was prepared from a blood sample contributed by each study participant using a non-organic purification method. Genotyping with Affymetrix GeneChip Human Mapping 500K Array Sets was performed following the protocol provided by the Manufacturer (Affymetrix, Santa Clara, Calif.). Briefly, an aliquot of the patient's DNA was prepared for hybridization to microarrays of SNPs in a series of reactions. The pattern in which patient DNA hybridized to the microarrays indicated the patient's genotype at each of 500,000 SNPs.

Quality assessment consisted of evaluating Hardy-Weinberg equilibrium (HWE) for each SNP, and the overall call rate for SNPs on a single chip less than 85% were eliminated from the analysis. A p-value threshold of 0.001 was used to identify SNPs not in HWE. Deviations from HWE may be caused by genuine associations or genotyping errors. Thus all SNPs determined not to be in HWE were manually inspected for evidence of genotyping errors. Association at a SNP was determined when allele frequencies were significantly different in the case population relative to the control population (see Table 2 above). Association was determined by performing a standard chi-squared test using allele frequencies between cohorts (control and AMD).

Two normative data sets were utilized as a control to confirm the phenotype was disease-associated. The first was the HapMap CEU population which is comprised of Centre d′ Etude du Polymorphisme Humain (CEPH) individuals of Caucasian ethnicity (see The International HapMap Consortium, 2003, Nature 426:789-796; and Thorisson et al., 2005, Genome Res. 15:1592-1593). Population-based allele frequencies for each SNP were provided as part of the annotations available for the Affymetrix genotyping platforms. This population provided a representative sample of the general population, and was therefore expected to have the population prevalence of glaucoma within that population. The second normative population used was a disease-free set of 100 patients drawn from the University of Iowa Ophthalmology clinic. These patients were all over the age of 59 at the time of ascertainment, and had no signs or history of AMD.

The analysis was performed on each SNP and contiguous regions using multiple SNPs. A region of interest was identified as comprising any single SNP (and the surrounding genomic sequence) that was associated with the phenotype in the disease population, and that showed no bias in the control population. Additionally, further strength of signal was indicated by: (1) more severe deviation of allele frequencies in case versus control, and (2) multiple SNPs that were all associated and clustered in a locus.

Regions of interest were identified based on the number of associated SNPs. SNP's were first clustered such that a cluster had to have at least three SNPs with p-values less than or equal to 1×10⁻⁴. Clusters having two SNPs adjacent to each other and separated by no more than 200 kb nucleotides were selected as relevant for diagnosis of AMD or AMD risk. The p-values, which are probabilities of the test statistic having a value at least as extreme as the value actually observed, were determined by the chi-square test.

The experiment was conducted in two phases, Phase 1A and Phase 1B. Phase 1A consisted of 200 patients with AMD and 200 patients without AMD. These 400 patients were each genotyped at 500,000 single nucleotide polymorphisms (SNPs) using 500K SNP chips from Affymetrix (Santa Clara, Calif.). Phase 1B consisted of another 200 patients with AMD and 200 patients without AMD. The results of Phase 1B were used to confirm the results from Phase 1A.

An additional phase was conducted to determine if the results from the compilation of Phase 1A and 1B could be replicated. This confirmatory phase was conducted by comparing the results from Phase 1A/1B with a second round of genotyping called “Phase 2.” The Phase 2 experiment consisted of genotyping additional subjects diagnosed with glaucoma, AMD as well as normal “controls.” The control samples validated that the signals from Phase 1A/1B were associating with the appropriate disease (glaucoma or AMD). An additional 460 AMD subjects, 230 glaucoma samples and 368 “control” samples were genotyped. The samples were combined into pools of 46 samples and the pools were genotyped in duplicate with the Affymetrix 5.0 genotype mapping arrays. Allele frequencies within the pools were estimated based upon the relative allelic intensities of the allele-specific probesets on the genotyping arrays. The results of Phase 2 are shown in Table 1 as data.AMD-GLC (comparison between AMD and glaucoma samples), data.AMD-NL (comparison between AMD and normal samples), and data.GLC-NL (comparison between glaucoma and normal samples).

### AMD Loci

The genome-wide association analysis identified four AMD-associated loci, as shown in Table 1 above, and in Table 3 below.

Of the four AMD-associated loci identified, two were the previously reported AMD loci CFH and chromosome 10. The chromosome 10 locus contains the PLEKHA1, ARMS2 (LOC387715) and HTRA1 genes. The strongest association appeared to be the Arg69Ser variation in ARMS2, which was reported by Kanda et al. (2007, Proc. Natl. Acad. Sci. USA 9:16227-32). This study also identified two novel AMD-associated loci with suggestive p-values, as shown in Table 3.

TABLE 3

| | | | Joint p- | p-value | p-value | |
|---|---|---|---|---|---|---|
| Position | SNPs^{a} | Best SNP | value | (primary) | (replicate) | Genes |
| chr1 | 31 | SNP_A- | <10⁻¹⁶ | 6.67E−13 | 1.17E−08 | KCNT2, CFH, CFHR3, |
| 194664398- | | 2171106 | | | | CFHR1P, CFHR4, |
| 195393272 | | | | | | CFHR2, CFHR5, F13B, |
| | | | | | | ASPM, ZBTB41 |
| chr10 | 7 | SNP_A- | <10⁻¹⁶ | 1.11E−07 | 3.07E−11 | PLEKHA1, ARMS2, |
| 124139342- | | 1841655 | | | | HTRA1 |
| 124225345 | | | | | | |
| chr8 | 4 | SNP_A- | 1.50E−04 | 1.09E−02 | 5.82E−03 | No genes in region |
| 139046190- | | 4195154 | | | | |
| 139064822 | | | | | | |
| chr6 | 5 | SNP_A- | 2.17E−04 | 2.45E−03 | 3.12E−02 | DDR1, GTF2H4, |
| 30911233- | | 2196694 | | | | VARS2, SFTA2, |
| 31030549 | | | | | | DPCR1 |

^{a}Number of associated SNPs.

The first novel AMD locus was located on chromosome 8, containing four associated SNPs with a peak p-value of 1.5×10⁻⁴. There were no annotated genes within this locus. A second novel AMD locus was located on chromosome 6 located 34 kb upstream of the DDR1 gene, containing five associated SNPs with a peak p-value of 2.17×10⁻⁴. Four other genes were also found within this locus, including GTF2H4, VARS2, SFTA2 and DPCR1.

A complication of AMD is choroidal neovascularization (CNV), commonly known as “wet” or exudative AMD. In addition to looking for AMD associations, AMD patients with and without CNV were compared to look for potential risk-associated or protective loci. This analysis identified a suggestive, reproducible locus on chromosome 10 between rs1444775 and rs1361777 that had a peak p-value of 3.2×10⁻⁵. No genes were identified within or near this region.

A set of previously reported AMD causing or AMD risk associated loci (Daiger et al., 1998, IOVS, 5295) were also evaluated. The analysis of these loci is presented in Table 4. Three of the loci (BSMD, MCDR3 and MCDR4) were associated with AMD in the primary and validation cohorts. The BSMD locus contained a cluster of associated SNPs with a peak p-value of 6.2×10⁻⁵. The associated SNPs localized within the first intron of the CAMK2A gene, a calcium-dependent serine/threonine kinase. The MCDR3 locus contained a cluster of eight associated SNPs with a peak p-value of 2×10⁻⁴. This associated locus contained the CCT5 gene, part of the chaperonin containing TCP1 complex, and the first exon of the FAM173B gene. The MCDR4 locus contained three associated SNPs with a peak p-value of 0.002. There were no annotated genes within the MCDR4 associated SNP cluster.

TABLE 4
Evidence for association to linked AMD loci.

| Linked | Peak | Primary | Validation |
|---|---|---|---|
| Locus | p-value | p-value | p-value |
| BCMAD | 1.1E−06 | 8.0E−01 | 1.12E−10 |
| BSMD | 6.2E−05 | 4.9E−03 | 5.1E−03 |
| MCDR1 | 3.4E−04 | 1.0E−04 | 8.2E−01 |
| MCDR3 | 2.5E−04 | 2.4E−02 | 4.0E−03 |
| MCDR4 | 2.9E−03 | 4.1E−02 | 3.4E−02 |
| MCDR5 | 3.3E−03 | 2.2E−04 | N/A |
| MDDC | 5.4E−08 | 6.6E−01 | 1.2E−11 |
| TEAD1 | 1.2E−02 | 4.9E−02 | 1.2E−01 |

### Example 2

### Interactions Among AMD Loci

Genetic interactions within the CFH and ARMS2 loci were assessed in a genome-wide fashion. In agreement with previous work, no genetic interaction was found between the risk alleles of CFH and ARMS2. No interactions were found to be significant after Bonferroni correction for multiple hypothesis testing. However, several suggestive associations (p<10⁻⁵) were identified—four to CFH and one to ARMS2. The interacting loci are listed in Table 5.

TABLE 5
Loci interacting with AMD risk alleles.

| Risk Allele | Peak p-value | SNPs^{a} | Genomic Position | Genes |
|---|---|---|---|---|
| ARMS2 | 1.0E−06 | 7 | chr8: 12,830,965-12,868,315 | C8ORF79 |
| CFH | 2.2E−06 | 3 | chr1: 145,409,110-145,569,478 | BCL9 |
| CFH | 3.0E−06 | 4 | chr12: 2,419,279-2,428,736 | CACNA1C |
| CFH | 6.2E−06 | 3 | chr17: 36,871,580-36,887,675 | KRT32 |
| CFH | 1.0E−05 | 4 | chr9: 78,205,224-78,296,472 | GCNT1 |

^{a}Number of associated SNPs.

The first locus interacting with the CFH Y402H risk allele was a region on chromosome 1 containing three associated SNPs with a peak p-value of 2.2×10⁻⁶. This region spans the BCL9 gene. The second locus interacting with the CFH risk allele was a region on chromosome 12. This region contained four associated SNPs found within the third intron of CACNA1C, with a peak p-value of 3.0×10⁻⁶. Another CFH-interacting locus was found within the initial intron of the GCNT1 gene on chromosome 9. This interaction was supported by four associated SNPs with a peak p-value of 1.0×10⁻⁵. This locus contained the initial exon of the GCNT1 gene. The SNP clusters associated with CFH are shown in Table 6, and the allele frequency of the associated SNP clusters are shown in Table 7.

TABLE 6
CFH SNP Clusters

| | |
|---|---|
| SNP | P-Value |

Cluster CFH(1)
Chromosome: chr1, Genomic Start: 145409110,
Genomic Stop: 145569478

| | |
|---|---|
| SNP_A-2151688 | 2.27E−06 |
| SNP_A-2185264 | 2.17E−05 |
| SNP_A-2030977 | 8.58E−04 |

Cluster CFH(2)
Chromosome: chr12, Genomic Start: 2419279, Genomic Stop: 2428736

| | |
|---|---|
| SNP_A-1900681 | 3.03E−06 |
| SNP_A-1873965 | 2.14E−05 |
| SNP_A-4285500 | 3.69E−04 |
| SNP_A-2110586 | 6.42E−04 |

Cluster CFH(3)
Chromosome: chr17, Genomic Start: 36871580, Genomic Stop: 36887675

| | |
|---|---|
| SNP_A-2060952 | 6.21E−06 |
| SNP_A-1908709 | 1.81E−04 |
| SNP_A-2187866 | 9.75E−04 |

Cluster CFH(4)
Chromosome: chr9, Genomic Start: 78205224, Genomic Stop: 78296472

| | |
|---|---|
| SNP_A-2268126 | 1.06E−05 |
| SNP_A-1948519 | 3.04E−05 |
| SNP_A-2037400 | 3.00E−04 |
| SNP_A-4298636 | 7.22E−04 |

TABLE 7
CFH SNP Cluster Allele Frequency

| | | Allele Frequency | | | | | | |
|---|---|---|---|---|---|---|---|---|
| | | AMD | | | GLC | | | |
| SNP Name | Allele | AA | AB | BB | AA | AB | BB | P-Value |
| Cluster CFH(1) | | | | | | | | |
| SNP_A-2151688 | [C/T] | 44 | 88 | 62 | 41 | 99 | 47 | 2.27E−06 |
| SNP_A-2185264 | [C/T] | 58 | 88 | 39 | 50 | 91 | 36 | 2.17E−05 |
| SNP_A-2030977 | [C/G] | 247 | 128 | 17 | 248 | 119 | 21 | 8.58E−04 |
| Cluster CFH(2) | | | | | | | | |
| SNP_A-1900681 | [A/G] | 17 | 112 | 265 | 16 | 121 | 256 | 3.03E−06 |
| SNP_A-1873965 | [A/C] | 17 | 131 | 218 | 15 | 141 | 187 | 2.14E−05 |
| SNP_A-4285500 | [C/T] | 233 | 140 | 23 | 230 | 142 | 24 | 3.69E−04 |
| SNP_A-2110586 | [C/G] | 238 | 133 | 26 | 228 | 144 | 24 | 6.42E−04 |
| Cluster CFH(3) | | | | | | | | |
| SNP_A-2060952 | [C/T] | 193 | 166 | 19 | 164 | 179 | 22 | 6.21E−06 |
| SNP_A-1908709 | [G/T] | 253 | 132 | 8 | 276 | 104 | 7 | 1.81E−04 |
| SNP_A-2187866 | [G/T] | 12 | 130 | 256 | 12 | 106 | 281 | 9.75E−04 |
| Cluster CFH(4) | | | | | | | | |
| SNP_A-2268126 | [C/T] | 148 | 189 | 62 | 148 | 185 | 65 | 1.06E−05 |
| SNP_A-1948519 | [C/G] | 153 | 187 | 59 | 153 | 189 | 55 | 3.04E−05 |
| SNP_A-2037400 | [C/G] | 0 | 71 | 124 | 0 | 63 | 126 | 3.00E−04 |
| SNP_A-4298636 | [C/G] | 38 | 161 | 196 | 33 | 153 | 205 | 7.22E−04 |

The single ARMS2-interacting locus contained seven SNPs in a cluster on chromosome 8 spanning 38 kb, with a peak p-value of 1.02×10⁻⁶. These SNPs all lie within or upstream (within 15 kb) of the C8ORF79 gene. This gene was predicted to have methyltransferase activity based upon domain structure, but is otherwise uncharacterized. The SNP clusters associated with ARMS2 are shown in Table 8, and the allele frequency of the associated SNP clusters are shown in Table 9.

TABLE 8
ARMS2 SNP Cluster
Cluster ARMS2(1)
Chromosome: chr8, Genomic Start: 12830965, Genomic Stop: 12868315

| SNP | P-Value |
|---|---|
| SNP_A-2182313 | 1.03E−06 |
| SNP_A-2029196 | 4.06E−05 |
| SNP_A-1796946 | 7.94E−05 |
| SNP_A-1889641 | 8.07E−05 |
| SNP_A-2305126 | 1.55E−04 |
| SNP_A-1803016 | 2.22E−04 |
| SNP_A-2150207 | 4.01E−04 |

TABLE 9
ARMS2 SNP Cluster Allele Frequency

| | | Allele Frequency | | | | | | |
|---|---|---|---|---|---|---|---|---|
| Cluster ARMS2(1) | | AMD | | | GLC | | | |
| SNP Name | Allele | AA | AB | BB | AA | AB | BB | P-Value |
| SNP_A-2182313 | [C/T] | 47 | 164 | 181 | 44 | 151 | 191 | 1.03E−06 |
| SNP_A-2029196 | [A/T] | 40 | 167 | 189 | 39 | 138 | 217 | 4.06E−05 |
| SNP_A-1796946 | [G/T] | 106 | 204 | 84 | 120 | 191 | 85 | 7.94E−05 |
| SNP_A-1889641 | [C/G] | 88 | 192 | 105 | 89 | 173 | 114 | 8.07E−05 |
| SNP_A-2305126 | [A/G] | 83 | 205 | 107 | 93 | 178 | 121 | 1.55E−04 |
| SNP_A-1803016 | [A/C] | 82 | 203 | 108 | 88 | 181 | 123 | 2.22E−04 |
| SNP_A-2150207 | [C/T] | 98 | 175 | 116 | 85 | 197 | 99 | 4.01E−04 |

### Example 3

### SNPs Associated with BCMAD and BSMD Loci

The data generated in the studies discussed above were used to identify AMD-associated SNPs present in the BCMAD and BSMD loci. Tables 10 and 11 show the SNPs that are associated with AMD that also reside in the previously published regions linked to AMD. These SNPs indicate regions of the genome that are associated with increased risk of developing AMD, and may define the genes harboring the Mendelianly-segregating mutations in the BCMAD and BSMD loci

TABLE 10
Associated SNPs in BCMAD Loci

| | | Joint | data.AMD- | data.AMD- |
|---|---|---|---|---|
| SNP | Position | p-value | GLC | NL |
| SNP_A-4211633 | 48128811 | 3.50E−02 | 0.031402422 | 0.026806612 |
| SNP_A-2262048 | 51711636 | 4.60E−02 | 0.030796637 | 0.026814696 |
| SNP_A-2144053 | 51951501 | 9.30E−03 | 0.002777989 | 0.003471191 |
| SNP_A-2127298 | 51952164 | 2.60E−02 | 0.029218217 | 0.009281392 |
| SNP_A-1861884 | 56282172 | 5.00E−02 | 0.04305168 | 0.005012254 |
| SNP_A-4265708 | 67924742 | 4.30E−02 | 0.000959293 | 0.000380255 |
| SNP_A-4265756 | 80232118 | 3.70E−02 | 0.015957837 | 0.008212006 |
| SNP_A-2036332 | 85456156 | 3.80E−02 | 0.019012455 | 0.000299606 |
| SNP_A-2282217 | 90695684 | 4.00E−02 | 0.045583424 | 0.048876659 |
| SNP_A-1986845 | 92300776 | 4.10E−03 | 0.001695942 | 0.000407895 |

Table 11 shows the associated SNPS in the BSMD loci.

TABLE 11
Associated SNPs in BSMD Loci

| | | Joint | data.AMD- | data.AMD- |
|---|---|---|---|---|
| SNP | Position | p-value | GLC | NL |
| SNP_A-1882493 | 117226687 | 3.70E−02 | 0.00046495 | 0.01646974 |
| SNP_A-2311061 | 121438428 | 2.20E−02 | 0.00102768 | 0.01868334 |
| SNP_A-2286249 | 121505722 | 8.90E−03 | 0.00412525 | 0.01304729 |
| SNP_A-1983615 | 135420406 | 2.40E−02 | 0.00024435 | 0.00515232 |
| SNP_A-2110637 | 135679839 | 4.90E−02 | 0.00332581 | 0.03253993 |
| SNP_A-1840184 | 135689800 | 5.00E−02 | 0.00653768 | 0.04148296 |
| SNP_A-1983622 | 135724949 | 2.30E−02 | 0.01653463 | 0.22525222 |
| SNP_A-1983623 | 135737176 | 4.00E−02 | 0.01385154 | 0.01585555 |

It should be understood that the foregoing disclosure emphasizes certain specific embodiments of the invention and that all modifications or alternatives equivalent thereto are within the spirit and scope of the invention as set forth in the appended claims.

What is claimed is:

1. A method for diagnosing increased risk of AMD in a patient, the method comprising:
a) obtaining a biological sample containing nucleic acid from the patient; and
b) analyzing the nucleic acid to detect the presence or absence of a AMD-associated polymorphism identified in Table 1, 6, 8, 10, or 11
wherein the presence of a AMD-associated polymorphism identified in Table 1, 6, 8, 10, or 11 indicates an increased risk for AMD.

2. The method of claim 1, wherein the AMD-associated polymorphism is a single nucleotide polymorphism (SNP) identified in Table 1, 6, 8, 10 or 11, and having a P-value of less than 1×10⁻⁴.

3. The method of claim 2, wherein the presence of at least two SNPs identified in Table 1, 6, 8, 10 or 11 indicates an increased risk for AMD.

4. The method of claim 1, wherein the AMD-associated polymorphism is a single nucleotide polymorphism cluster (SNP cluster) identified in Table 1, 6, 8, 10, or 11.

5. The method of claim 4, wherein the presence of at least two SNP clusters identified in Table 1, 6, 8, 10, or 11 correlates with an increased risk for AMD.

6. The method of claim 1, wherein analysis of the nucleic acid comprises allele specific primers or allele specific probes.

7. The method of claim 1, further comprising administering an agent suitable for treating AMD to a patient who has been identified as having an increased risk of AMD.

8. A microarray for determining AMD risk comprising a set of allele specific oligonucleotides capable of hybridizing to one or more of the AMD-associated polymorphisms identified in Table 1, 6, 8, 10, or 11.

9. A kit for determining whether a patient has an increased risk for AMD, comprising:
(a) at least one oligonucleotide that can identify a AMD-associated polymorphism identified in Table 1, 6, 8, 10, or 11; and
(b) instructions for use.

10. The kit of claim 9, comprising a set of oligonucleotides, wherein the set comprises at least one pair of primers that can detect at least one of the polymorphisms identified in Table 1, 6, 8, 10, or 11.

11. The kit of claim 10, wherein the set comprises a plurality of primer pairs, each of which can detect at least one single nucleotide polymorphism identified in Table 1, 6, 8, 10, or 11.

12. The kit of claim 10, wherein the set comprises a plurality of primer pairs, each of which can detect at least one SNP cluster identified in Table 1, 6, 8, 10, or 11.

13. The kit of claim 10, comprising a set of oligonucleotide probes, each of which can hybridize to a polymorphism identified in Table 1, 6, 8, 10, or 11.

14. The kit of claim 9, further comprising a microarray.
